(19) United States
(12) Patent Application Publication
(10) Pub. No.: US 2025/0248644 A1
(43) Pub. Date: Aug. 7, 2025

(54) METHOD FOR CALCULATING DEMENTIA-RELATED INDEX ON BASIS OF VOLUME OF EXTRACEREBRAL CEREBROSPINAL FLUID, AND ANALYSIS APPARATUS

(71) Applicant: SAMSUNG LIFE PUBLIC WELFARE FOUNDATION, Seoul (KR)

(72) Inventors: Sang Won SEO, Seoul (KR); Hye Min JANG, Seoul (KR); Yu Hyun PARK, Seoul (KR); Yuna GU, Seoul (KR)

(73) Assignee: SAMSUNG LIFE PUBLIC WELFARE FOUNDATION, Seoul (KR)

(21) Appl. No.: 19/189,782

(22) Filed: Apr. 25, 2025

(30) Foreign Application Priority Data
Oct. 28, 2022 (KR) 10-2022-0141232
Dec. 28, 2022 (WO) PCT/KR2022/021527

(51) Int. Cl. A61B 5/00; A61B 5/055; G06T 7/00; G06T 7/62

(52) U.S. Cl. CPC A61B 5/4088; A61B 5/0042; A61B 5/055; G06T 7/0012; G06T 7/62; G06T 2207/30016

(57) ABSTRACT

Proposed is a method of calculating a dementia-related index from a brain image, the method including the following steps of receiving, by an analysis apparatus, a brain image of a subject, identifying, by the analysis apparatus, a region of interest from the brain image, calculating, by the analysis apparatus, the volume or area of the region of interest, and calculating, by the analysis apparatus, a dementia-related index of the subject on the basis of the volume or area.

### CROSS REFERENCE TO RELATED APPLICATION

This application claims the benefit under 35 USC 119 (a) of Korean Patent Application No. 10-2022-0141232 filed on Oct. 28, 2022, in the K orean Intellectual Property Office, and PCT application No. PCT/KR2022/021527 filed on Dec. 28, 2022, in the World Intellectual Property Organization, the entire disclosure of which is incorporated herein by reference for all purposes.

### BACKGROUND

### 1. Field

The following description relates to a method of calculating dementia-related information on the basis of a brain image.

### 2. Description of the Related Art

Dementia refers to a syndrome causing impairment in cognitive functions such as memory, language, and judgment. Although there are many different types of dementia, Alzheimer's disease is the most common form of dementia.

Dementia is a progressive disease that develops over a long period of time, with pathological changes accumulating before the appearance of clinical symptoms. Thus, early diagnosis of dementia is critical in terms of delaying and managing the onset of dementia symptoms.

Brain images of magnetic resonance imaging (MRI), positron emission tomography (PET), and the like are used to diagnose dementia. Typically, cortical thickness is utilized as a dementia-related index.

### SUMMARY

In one general aspect, there is provided a method of calculating a dementia-related index based on the volume of an extracerebral cerebrospinal fluid region includes the following steps: receiving, by an analysis apparatus, a brain image of a subject; identifying, by the analysis apparatus, a region of interest from the brain image; calculating, by the analysis apparatus, the volume or area of the region of interest; and calculating, by the analysis apparatus, a dementia-related index of the subject on the basis of the volume or area.

In another aspect, there is provided a method of calculating a dementia-related index based on the volume of an extracerebral cerebrospinal fluid region includes the following steps: receiving, by an analysis apparatus, a brain image of a subject; identifying, by the analysis apparatus, a region of interest from the brain image; and inputting, by the analysis apparatus, the region of interest into a pre-trained learning model to calculate a dementia-related index of the subject.

In yet another aspect, there is provided an analysis apparatus configured to calculate a dementia-related index includes: an input device configured to receive a brain image of a subject; and a computing device configured to identify a region of interest from the brain image and calculate a dementia-related index on the basis of the region of interest. The region of interest includes an extracerebral cerebrospinal fluid region.

### BRIEF DESCRIPTION OF THE DRAWINGS

FIG. 1 illustrates an example of estimating cortical thickness in a medical image.

FIG. 2 illustrates an example of a system in which a dementia-related index is calculated by analyzing a brain image.

FIG. 3 illustrates an example of a process in which a dementia-related index is calculated by analyzing a brain image.

FIG. 4 illustrates an example of a process in which a dementia-related index is calculated on the basis of an extracerebral cerebrospinal fluid region and a ventricular region.

FIG. 5 shows evaluation results regarding the relevance of lateral ventricular and extracerebral cerebrospinal fluid regions to dementia assessment.

FIG. 6 shows evaluation results regarding the relevance of extracerebral cerebrospinal fluid subregions to dementia assessment.

FIG. 7 shows performance evaluation results of classifiers constructed on the basis of lateral ventricular and extracerebral cerebrospinal fluid regions.

FIG. 8 shows performance evaluation results of classifiers in which patient information is additionally applied to the model of FIG. 7.

FIG. 9 shows performance evaluation results of classifiers in which patient information is applied to lateral ventricular and extracerebral cerebrospinal fluid subregions.

FIG. 10 illustrates an example of a process in which a dementia-related index is calculated using a learning model.

FIG. 11 illustrates an example of an analysis apparatus configured to calculate a dementia-related index.

Throughout the drawings and the detailed description, unless otherwise described, the same drawing reference numerals should be understood to refer to the same elements, features, and structures. The sizes of regions and elements, and depiction thereof may be exaggerated for clarity, illustration, and/or convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be understood by those of ordinary skill in the art.

Moreover, descriptions of well-known functions and constructions may be omitted for increased clarity and conciseness. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. Unless stated otherwise, the same reference numerals may be used to refer to the same or substantially the same elements throughout the specification and the drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the embodiments described with reference to the accompanying drawings. The present disclosure may, however, be embodied in different forms and should not be construed as limited to the embodiments set forth herein. Rather, these embodiments are examples and are provided so that this disclosure may be thorough and complete to assist those skilled in the art to understand the inventive concepts without limiting the protected scope of the present disclosure.

Shapes, dimensions (e.g., sizes, lengths, locations, and areas), proportions, ratios, numbers, the number of elements, and the like disclosed herein, including those illustrated in the drawings, are merely examples, and thus, the present disclosure is not limited to the illustrated details. It is, however, noted that the relative dimensions of the components illustrated in the drawings are part of the present disclosure.

When the term “comprise,” “have,” “include,” “contain,” “constitute,” “made of,” “formed of,” “composed of,” or the like is used with respect to one or more elements (e.g., components, structures, groups, circuits, networks, members, parts, areas, portions, integers, steps, operations, and/or the like), one or more other elements may be added unless a term such as “only” or the like is used. The terms used in the present disclosure are merely used in order to describe particular example embodiments, and are not intended to limit the scope of the present disclosure. The terms of a singular form may include plural forms unless the context clearly indicates otherwise. For example, an element may be one or more elements. An element may include a plurality of elements. The word “exemplary” is used to mean serving as an example or illustration. Embodiments are example embodiments. Aspects are example aspects. In one or more implementations, “embodiments,” “examples,” “aspects,” and the like should not be construed to be preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

When a positional relationship between two elements (e.g., components, structures, groups, circuits, networks, members, parts, areas, portions, and/or the like) are described using any of the terms such as “adjacent to,” “beside,” “next to,” and/or the like indicating a position or location, one or more other elements may be located between the two elements unless a more limiting term, such as “immediate(ly),” “direct(ly),” or “close(ly),” is used. Furthermore, the spatially relative terms such as the foregoing terms as well as other terms such as “column,” “row,” “vertical,” “horizontal,” “diagonal,” and the like refer to an arbitrary frame of reference.

In describing a temporal relationship, when the temporal order is described as, for example, “after,” “following,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

It is understood that, although the terms “first,” “second,” and the like may be used herein to describe various elements (e.g., components, structures, groups, circuits, networks, members, parts, areas, portions, and/or the like), these elements should not be limited by these terms, for example, to any particular order, precedence, or number of elements. These terms are used only to distinguish one element from another. For example, a first element may denote a second element, and, similarly, a second element may denote a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element, and the like) are not limited by ordinal numbers or the names in front of the elements. Further, a first element may include one or more first elements. Similarly, a second element or the like may include one or more second elements or the like.

In describing elements of the present disclosure, the terms “first,” “second,” “A,” “B,” “(a),” “(b),” or the like may be used. These terms are intended to identify the corresponding element(s) from the other element(s), and these are not used to define the essence, basis, order, or number of the elements.

The expression that an element (e.g., component, structure, group, circuit, network, member, part, area, portion, and/or the like) “is engaged” with another element may be understood, for example, as that the element may be either directly or indirectly engaged with the another element. The term “is engaged” or similar expressions may refer to a term such as “is connected,” “is coupled,” “is combined,” “is linked,” “is provided,” “interacts,” or the like. The engagement may involve one or more intervening elements disposed or interposed between the element and the another element, unless otherwise specified.

The terms such as a “line” or “direction” should not be interpreted only based on a geometrical relationship in which the respective lines or directions are parallel, perpendicular, diagonal, or slanted with respect to each other, and may be meant as lines or directions having wider directivities within the range within which the components of the present disclosure may operate functionally.

The term “at least one” should be understood as including any and all combinations of one or more of the associated listed items. For example, each of the phrases “at least one of a first item, a second item, or a third item” and “at least one of a first item, a second item, and a third item” may represent (i) a combination of items provided by two or more of the first item, the second item, and the third item or (ii) only one of the first item, the second item, or the third item. Further, at least one of a plurality of elements can represent (i) one element of the plurality of elements, (ii) some elements of the plurality of elements, or (iii) all elements of the plurality of elements. Further, “at least some,” “at least some portions,” “at least some parts,” “at least a portion,” “at least one or more portions,” “at least a part,” “at least one or more parts,” “at least some elements,” “one or more,” or the like of a plurality of elements can represent (i) one element of the plurality of elements, (ii) a portion (or a part) of the plurality of elements, (iii) one or more portions (or parts) of the plurality of elements, (iv) multiple elements of the plurality of elements, or (v) all of the plurality of elements. Moreover, “at least some,” “at least some portions,” “at least some parts,” “at least a portion,” “at least one or more portions,” “at least a part,” “at least one or more parts,” or the like of an element can represent (i) a portion (or a part) of the element, (ii) one or more portions (or parts) of the element, or (iii) the element, or all portions of the element.

The expression of a first element, a second elements “and/or” a third element should be understood as one of the first, second and third elements or as any or all combinations of the first, second and third elements. By way of example, A, B and/or C may refer to only A; only B; only C; any of A, B, and C (e.g., A, B, or C); some combination of A, B, and C (e.g., A and B; A and C; or B and C); or all of A, B, and C. Furthermore, an expression “A/B” may be understood as A and/or B. For example, an expression “A/B” may refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element is referred to as being “between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as being different from one another. In another example, an expression “different from one another” may be understood as being different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and C.”

A phrase “substantially the same” may indicate a degree of being considered as being equivalent to each other taking into account minute differences due to errors in the manufacturing or operating process.

Features of various embodiments of the present disclosure may be partially or entirely coupled to or combined with each other, may be technically associated with each other, and may be variously operated, linked or driven together in various ways. Embodiments of the present disclosure may be implemented or carried out independently of each other or may be implemented or carried out together in a co-dependent or related relationship. In one or more aspects, the components of each apparatus and device according to various embodiments of the present disclosure are operatively coupled and configured.

The terms used herein have been selected as being general in the related technical field; however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example embodiments of the present disclosure are described in more detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for the convenience of description, a scale and dimension of each of the elements illustrated in the accompanying drawings may be different from an actual scale and dimension, and thus, embodiments of the present disclosure are not limited to a scale and dimension illustrated in the drawings. Before starting detailed explanations of figures, components that will be described in the specification are distinguished merely according to functions mainly performed by the components. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

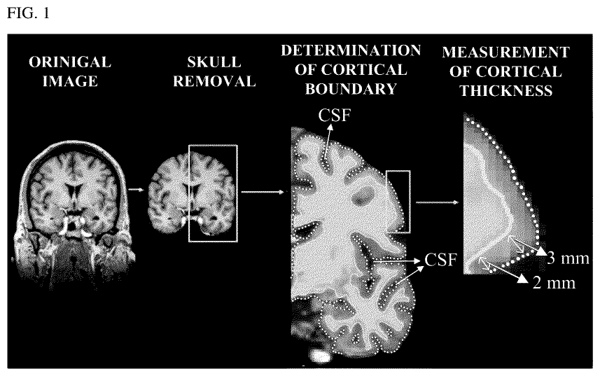

Alzheimer's disease is diagnosed on the basis of cortical thickness as identified in a medical image. FIG. 1 illustrates an example of estimating cortical thickness in a medical image. FIG. 1 illustrates an example of measuring or estimating cortical thickness in an MRI image. In FIG. 1, the cerebral cortex corresponds to the gray region between the solid white and dotted lines. Therefore, cortical thickness is accurately measurable only when the white region inside the solid white line and the gray region between the solid white and dotted lines are clearly distinguished. In the meantime, medical imaging equipment is provided by various vendors, and imaging parameters may vary depending on the equipment type, even among equipment manufactured by the same vendor.

The researchers collected data from the population that visited the affiliated medical institution (Samsung Seoul Hospital). The population includes 605 participants in a normal control group and 616 participants in a dementia group, those who were examined in Samsung Seoul Hospital between 2015 and 2021. All participants underwent dementia assessments, including brain M RI and amyloid PET. In this case, the normal control group consisted of amyloid-negative subjects, and the dementia group consisted of amyloid-positive subjects.

TABLE 1

| | | Measurement subject | | | | | |
|---|---|---|---|---|---|---|---|
| | | Aβ(−) NC | | | Aβ(+) ADD | | |
| | | Equipment type | | | | | |
| | | | | P | | | P |
| | | Achieva | Ingenia | value | Achieva | Ingenia | value |
| Population | N | 581 | 24 | | 588 | 28 | |
| size | | | | | | | |
| Cortical | Frontal | 3.16 ± 0.11 | 3.17 ± 0.09 | 0.530 | 3.01 ± 0.15 | 3.00 ± 0.12 | 0.762 |
| thickness | Parietal | 3.08 ± 0.11 | 3.08 ± 0.13 | 0.895 | 2.87 ± 0.20 | 2.86 ± 0.17 | 0.747 |
| | Temporal | 3.29 ± 0.11 | 3.38 ± 0.13 | 0.001 | 3.06 ± 0.18 | 3.06 ± 0.18 | 0.857 |
| | Occipital | 2.99 ± 0.16 | 3.08 ± 0.19 | 0.011 | 2.79 ± 0.19 | 2.78 ± 0.18 | 0.632 |
| | Global | 3.13 ± 0.11 | 3.17 ± 0.12 | 0.059 | 2.94 ± 0.15 | 2.93 ± 0.14 | 0.711 |

Table 1 shows the measurement results of cortical thickness of the subjects using Achieva and Ingenia, the imaging equipment from the same manufacturer (Philips) used in the medical institution to which the researchers belong. A β(−) NC refers to a normal control group without amyloid beta accumulation, and Aβ(+) ADD refers to an Alzheimer's disease patient group with amyloid beta accumulation. In Aβ(+) ADD, the cortical thickness obtained by MRI scan in the Achieva and Ingenia devices showed no significant difference. However, in Aβ(−) NC, the measurements obtained from the temporal and occipital lobes using the two devices showed significant differences. In other words, as the researchers expected, the measured cortical thickness was somewhat different when the medical devices were manufactured by the same vendor but differed in type. This is because there are differences in parameter values set for different imaging equipment.

FIG. 1 shows some regions of an extracerebral cerebrospinal fluid (extracerebral CSF) region. The extracerebral CSF region corresponds to a sulcus region between gyri. The extracerebral CSF region looks black or extremely dark in the M RI image. In other words, the extracerebral CSF region corresponds to a region that is visually distinctly distinguished from the gray region of the cerebral cortex on MRI. Therefore, it can be presumed that the extracerebral CSF region will be a region capable of being relatively accurately identified by segmentation models or image processing techniques of computer devices.

TABLE 2

| | | Measurement subject | | | | | |
|---|---|---|---|---|---|---|---|
| | | Aβ(−) NC | | | Aβ(+) ADD | | |
| | | Equipment type | | | | | |
| | | | | P | | | P |
| | | Achieva | Ingenia | value | Achieva | Ingenia | value |
| Population | N | 581 | 24 | | 588 | 28 | |
| size | | | | | | | |
| CSF | Lateral | 0.01 ± 0.01 | 0.01 ± 0.00 | 0.378 | 0.02 ± 0.01 | 0.02 ± 0.01 | 0.705 |
| space | ventricle | | | | | | |
| | Extracerebral | 0.46 ± 0.05 | 0.46 ± 0.05 | 0.742 | 0.52 ± 0.05 | 0.51 ± 0.04 | 0.285 |
| | CSF | | | | | | |

Table 2 shows measurement results of the length (or size) of the extracerebral CSF region for the population described in Table 1, using the A chieva and Ingenia devices. From a detailed look at the results in Table 2, the length of the extracerebral CSF region was found to show no significant difference between the two devices in both Aβ(+) ADD and Aβ(−) NC. In other words, as the researchers predicted, it can be presumed that the extracerebral CSF region has distinct imaging features compared to the cerebral cortical region. Cortical thickness is an index for the degree of brain atrophy. The more severe the degree of brain atrophy, the smaller the cortical thickness. In addition, the size of the extracerebral CSF region correlates with the degree of brain atrophy. The more severe the degree of brain atrophy, the larger the size of the extracerebral CSF region.

Hereinafter, dementia refers to Alzheimer's dementia.

The following technology to be described is a technology for diagnosing dementia or predicting the possibility of developing dementia on the basis of the extracerebral CSF region.

The size of the extracerebral CSF region may be assessed as the gap or distance between gyri. Alternatively, the size of the extracerebral CSF region may be assessed as the area of a specific sulcus. The size of the area may be calculated from a two-dimensional (2D) image. Alternatively, the size of the extracerebral CSF region may be assessed as the volume of a specific sulcus. The volume may be calculated from a three-dimensional (3D) image or 2D slices.

Hereinafter, an apparatus configured to calculate a dementia-related index of a subject by analyzing a brain image is referred to as an analysis apparatus. The analysis apparatus may have forms of a computer device, such as a personal computer (PC), a smart device, a network server, a chipset dedicated to data processing, and the like.

The analysis apparatus may calculate the size or volume of a specific extracerebral CSF region in a brain image using a conventional image processing technique. Alternatively, the analysis apparatus may calculate the size or volume of a specific extracerebral CSF region using a deep learning network-type model. The analysis apparatus may calculate the degree of brain atrophy on the basis of the size or volume of the extracerebral CSF region. Furthermore, the analysis apparatus may produce a diagnosis or prediction results of dementia for the subject by analyzing a brain image.

The analysis apparatus calculates a dementia-related index by analyzing a brain image. In this case, the dementia-related index corresponds to an index or information related to dementia progression. The dementia-related index may include at least one piece of information including the size or volume of the extracerebral CSF region, the degree of brain atrophy assessed by the size or volume of the extracerebral CSF region, and the presence or absence of dementia (or the degree of dementia progression) assessed by the size/volume (or the degree of brain atrophy) of the extracerebral CSF region.

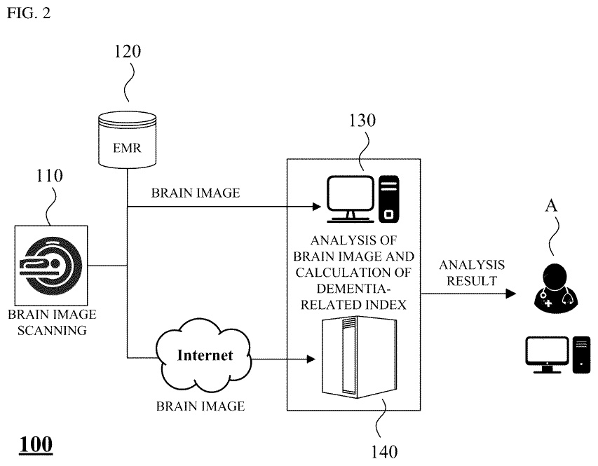

FIG. 2 illustrates an example of a system 100 in which a dementia-related index is calculated by analyzing a brain image. In FIG. 2, a computer terminal 130 and a server 140 are illustrated as examples of the analysis apparatus.

Medical imaging equipment 110 produces a brain image (for example, an M RI image) of a patient. The brain image produced by the medical imaging equipment 110 may be stored in a separate database such as an electronic medical record (EM R) 120.

In FIG. 2, a user A may analyze the brain image using the computer terminal 130 to obtain the dementia-related index. The computer terminal 130 may receive a brain image of a specific subject from the medical imaging equipment 110 or the EM R 120 via a wired or wireless network. In some cases, the computer terminal 130 may be an apparatus physically connected to the medical imaging equipment 110. The computer terminal 130 extracts an extracerebral CSF region from the brain image. The computer terminal 130 may calculate the dementia-related index on the basis of the extracerebral CSF region. (i) The computer terminal 130 may estimate the size or volume of the extracerebral CSF region. (ii) The computer terminal 130 may estimate the degree of brain atrophy depending on the size or volume of the extracerebral CSF region. (iii) The computer terminal 130 may estimate the presence or absence of dementia, the possibility of developing dementia, the degree of dementia progression, or the like, depending on the size/volume of the extracerebral CSF region or the degree of brain atrophy. The user A may confirm the analysis results on the computer terminal 130.

The server 140 may receive a brain image of a specific subject from the medical imaging equipment 110 or the EM R 120. The server 140 extracts an extracerebral CSF region from the brain image. The server 140 may calculate the dementia-related index on the basis of the extracerebral CSF region. (i) The server 140 may estimate the size or volume of the extracerebral CSF region. (ii) The server 140 may estimate the degree of brain atrophy depending on the size or volume of the extracerebral CSF region. (iii) The server 140 may estimate the presence or absence of dementia, the possibility of developing dementia, the degree of dementia progression, or the like, depending on the size/volume of the extracerebral CSF region or the degree of brain atrophy. The server 140 may transmit the analysis results of the brain image to a terminal of the user A. The user A may confirm the analysis results through the user terminal.

The computer terminal 130 and/or the server 140 may also store the analysis results in the EM R 120.

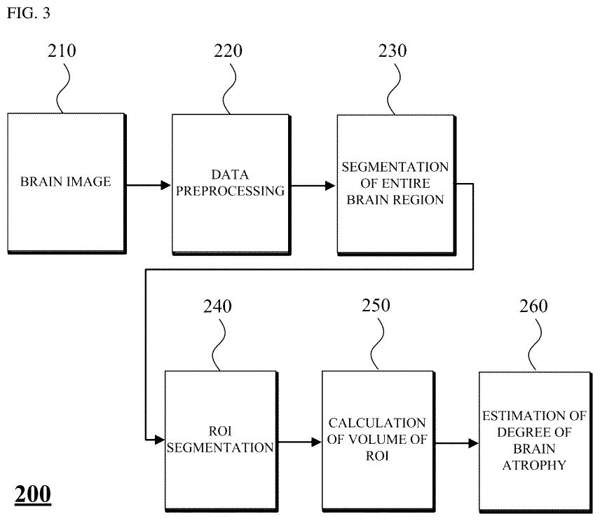

FIG. 3 illustrates an example of a process 200 in which a dementia-related index is calculated by analyzing a brain image.

The analysis apparatus receives a brain image (M RI image) of a subject (210).

The analysis apparatus may consistently preprocess data of the received brain image (220). Preprocessing the data may be a process of extracting surface structures or models of brain structures from the received image to produce a mask. For example, the analysis apparatus may extract the entire brain region from a brain MRI image using the CIVET pipeline. The analysis apparatus may extract brain regions from MRI slices. The analysis apparatus may extract brain regions from continuous M RI slices to extract 3D brain regions. To sum up, (i) the analysis apparatus may produce a mask of the entire brain region from the brain image. In addition, (ii) the analysis apparatus may also produce a mask of a specific region from the brain image. The specific region may include at least one of an extracerebral CSF region, extracerebral CSF subregions, a ventricular region, and ventricular subregions. The extracerebral CSF subregions and the ventricular subregions are to be described later.

In the meantime, the data preprocessing process for mask production may be an optional process. Among image processing techniques, when using a technique to automatically extract a region of interest (ROI), the analysis apparatus may not provide the mask in advance.

The analysis apparatus may segment the entire brain region from the brain image of the subject (230). The analysis apparatus may segment the entire brain region using the mask provided during the data preprocessing process. Alternatively, the analysis apparatus may segment the entire brain region using a segmentation model.

The analysis apparatus may segment the ROI from the entire brain region (240). The analysis apparatus may segment the ROI using the mask provided during the data preprocessing process. Alternatively, the analysis apparatus may segment the ROI using a segmentation model. The ROI includes the extracerebral CSF region. The ROI may include other regions in addition to the extracerebral CSF region. In addition, the ROI may be composed of subregions capable of being extracted from the extracerebral CSF region. Specific ROI is to be described later.

The analysis apparatus may calculate the volume of the segmented ROI (250). The analysis apparatus may calculate the volume of the 3D ROI using commonly used programs or algorithms. In the meantime, the analysis apparatus may calculate the area of the 2D ROI from an MRI slice. The analysis apparatus may calculate the area of the ROI from all slices or a specific slice selected.

The analysis apparatus may estimate the degree of brain atrophy on the basis of the volume or area of the ROI (260). The dementia-related index in FIG. 3 is the degree of brain atrophy. The volume of the ROI or the area of a specific point (or points) has a consistent correlation with the degree of brain atrophy. The correlation between the volume (or area) of the ROI and the degree of brain atrophy may be provided in advance in a table form. In this case, the analysis apparatus may estimate the degree of brain atrophy of the subject on the basis of the calculated volume (or area) of the ROI. Alternatively, the analysis apparatus may estimate the degree of brain atrophy of the subject using a function with the calculated volume (or area) of the ROI as a variable. This function may be a known formula. Alternatively, this function may be a formula provided in advance through regression analysis.

The ROI includes the extracerebral CSF region. Furthermore, the ROI may include at least some of the individual subregions of the extracerebral CSF region. In addition, the ROI may also include a ventricular region that is neither white nor gray on MRI and thus is relatively distinctly distinguished. Moreover, the ROI may include at least some of the individual subregions of the ventricular region. The ROI may be one of several regions or any one of several possible combinations thereof. ROI candidates are as listed in Table 3 below.

TABLE 3

| Entire region | Subregions |
|---|---|
| 1. Extracerebral CSF region | 1-1. Frontal region |
| | 1-2. Temporal region |
| | 1-3. Parietal region |
| | 1-4. Occipital region |
| 2. Ventricular region | 2-1. Lateral ventricle |
| | 2-2. Third ventricle |
| | 2-3. Fourth ventricle |

Possible ROIs are as follows. (i) The ROI may be at least one of the entire extracerebral CSF region and the entire ventricular region. (ii) In addition, the ROI may be at least one of the entire extracerebral CSF region and the ventricular subregions. (iii) In addition, the ROI may be at least one of the extracerebral CSF subregions and the entire ventricular region. (iv) In addition, the ROI may be at least one of the extracerebral CSF subregions and the ventricular subregions.

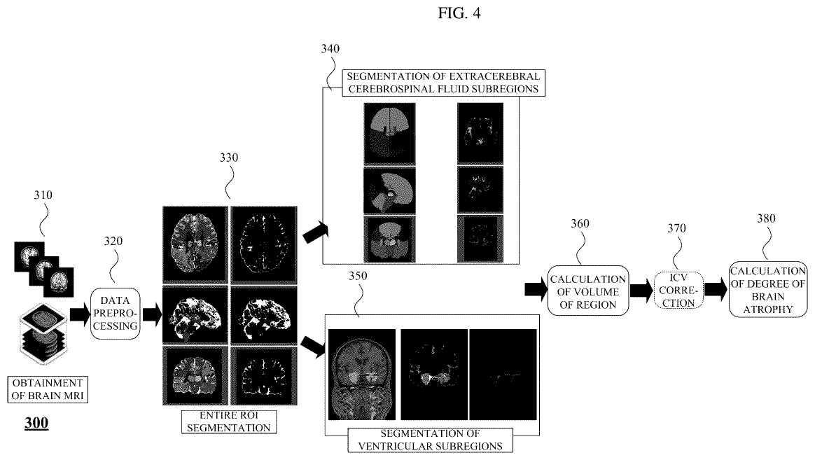

FIG. 4 illustrates an example of a process 300 in which a dementia-related index is calculated on the basis of an extracerebral CSF region and a ventricular region. FIG. 4 illustrates an example of calculating the dementia-related index using the subregions among ROIs.

The analysis apparatus receives a brain image (M RI image) of a subject (310).

The analysis apparatus may consistently preprocess data of the received brain image (320). Preprocessing the data may be a process of extracting surface structures or models of brain structures or models from the received image to produce a mask. The analysis apparatus may produce a mask of the entire brain region from the brain image. In addition, the analysis apparatus may also produce a mask of a specific region from the brain image. The specific region may include at least one of the extracerebral CSF region, the extracerebral CSF subregions, the ventricular region, and the ventricular subregions.

In the meantime, the data preprocessing process for mask production may be an optional process. Among image processing techniques, when using a technique to automatically extract the ROI, the analysis apparatus may not provide the mask in advance. The analysis apparatus may segment the entire ROI region from the brain image of the subject (330). The analysis apparatus may segment the entire brain region using the mask provided during the data preprocessing process. Alternatively, the analysis apparatus may segment the entire brain region using a segmentation model.

The analysis apparatus may segment a specific ROI from the entire brain region. The analysis apparatus may segment at least one of the extracerebral CSF subregions using the mask (340). In addition, the analysis apparatus may segment at least one of the ventricular subregions using the mask (350). The analysis apparatus may segment a target ROI using a segmentation model.

The analysis apparatus may calculate the volume of the segmented ROI (360). The analysis apparatus may calculate the volume of the 3D ROI using commonly used programs or algorithms. In the meantime, the analysis apparatus may calculate the area of the 2D ROI from an MRI slice. The analysis apparatus may calculate the area of the ROI from all slices or a specific slice selected.

The analysis apparatus may normalize each brain region using the intracrani al volume (ICV) of the subject (370). The analysis apparatus may consistently correct the size or volume of the target ROI on the basis of the ICV. The ICV-based correction process may be an optional process.

The analysis apparatus may estimate the degree of brain atrophy on the basis of the volume or area of the ROI (380). The analysis apparatus may estimate the degree of brain atrophy on the basis of the volume or area corrected on the basis of the ICV (380). For example, the analysis apparatus may normalize the volume by dividing the volume (or area) of the ROI by the ICV.

The dementia-related index in FIG. 4 is the degree of brain atrophy. The analysis apparatus may estimate the degree of brain atrophy of the subject by comparing the calculated volume (or area) of the ROI and a reference provided in advance. Alternatively, the analysis apparatus may estimate the degree of brain atrophy of the subject using a function with the calculated volume (or area) of the ROI as a variable.

The researchers selected the ROI for calculating the dementia-related index.

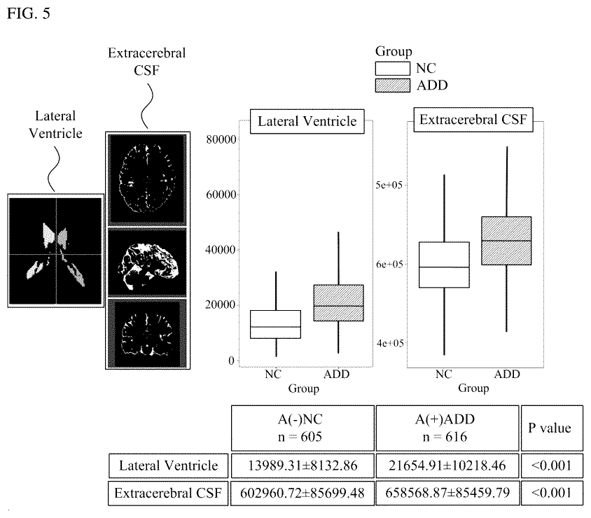

The researchers identified the relevance of the lateral ventricles of the ventricular subregions and the entire extracerebral CSF region to dementia. FIG. 5 shows evaluation results regarding the relevance of the lateral ventricular and extracerebral CSF regions to dementia assessment. In FIG. 5, A (−) NC refers to a normal control group without amyloid beta accumulation, and A (+) ADD refers to an Alzheimer's disease patient group with amyloid beta accumulation. From a detailed look at the results in FIG. 5, each of the lateral ventricular and extracerebral CSF regions may be the ROI. (i) The lateral ventricular region may be an index enabling the normal control group and the patient group to be distinguished (p<0.001). In addition, (ii) the entire extracerebral CSF region may also be an index enabling the normal control group and the patient group to be distinguished (p<0.001).

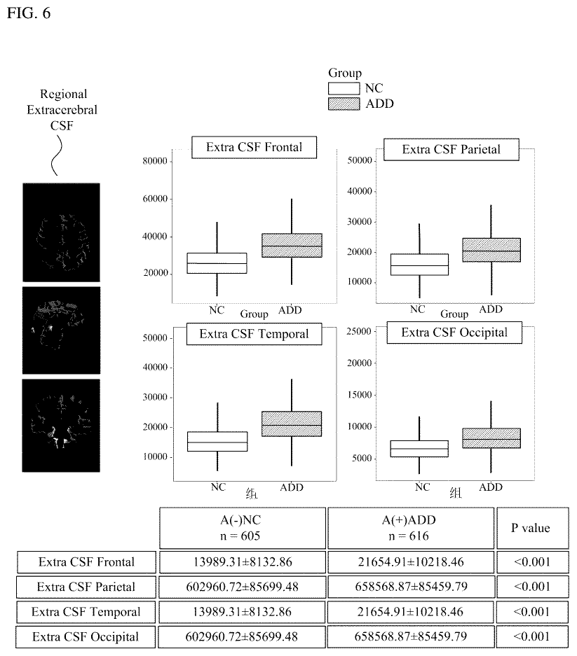

The researchers identified the relevance of each of the extracerebral CSF subregions to dementia. FIG. 6 shows evaluation results regarding the relevance of the extracerebral CSF subregions to dementia assessment. The extracerebral CSF subregions include the frontal region (F), the temporal region (T), the parietal region (P), and the occipital region (O). In FIG. 6, A (−) NC refers to a normal control group without amyloid beta accumulation, and A (+) ADD refers to an Alzheimer's disease patient group with amyloid beta accumulation. From a detailed look at the results in FIG. 6, each of the extracerebral CSF subregions may be the ROI (p<0.001).

The researchers constructed a model (classifier) to calculate the dementia-related index on the basis of the selected ROI. The classifier was implemented as a machine learning model. The researcher used 70% of the data from the population described above as learning data and the remainder of 30% as verification data. The researchers performed logistic regression using glm( ) in R. However, the classifier may also be implemented as other models, such as deep learning models.

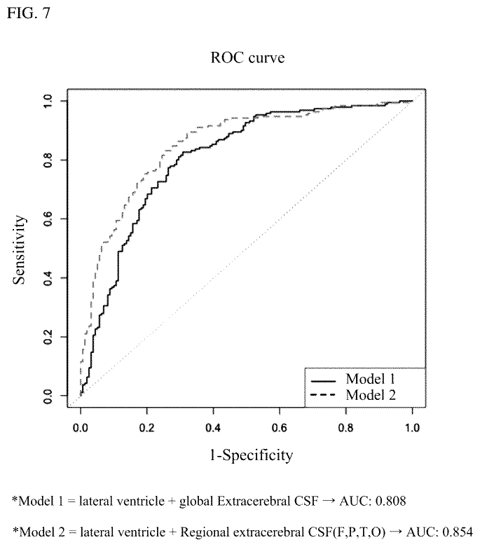

FIG. 7 shows performance evaluation results of classifiers constructed on the basis of the lateral ventricular and extracerebral CSF regions. FIG. 7 shows the performance of two models (Model 1 and Model 2). Model 1 and Model 2 are models to calculate the dementia-related index using only brain images. Model 1 is a model that uses the lateral ventricular and entire extracerebral CSF regions as the ROIs. Model 2 is a model that uses the lateral ventricular and extracerebral CSF subregions as the ROIs. The area under the receiver operating characteristic (ROC) curve (AUC) of Model 1 was 0.808. The subregions of the extracerebral CSF region used in Model 2 are the frontal region (F), the temporal region (T), the parietal region (P), and the occipital region (O). The AUC of Model 2 was 0.854. Although Model 2 slightly outperformed Model 1, both Model 1 and Model 2 were found to be sufficiently meaningful in diagnosing or predicting dementia.

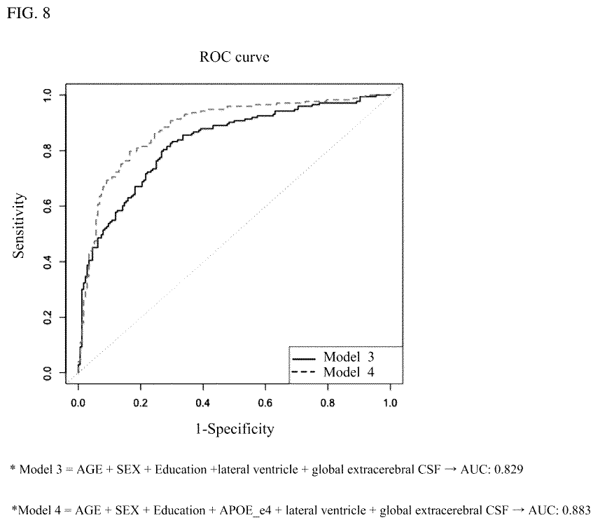

FIG. 8 shows performance evaluation results of classifiers in which patient information is additionally applied to the lateral ventricular and entire extracerebral CSF regions. FIG. 8 shows the performance of two models (Model 3 and Model 4). Model 3 and Model 4 are models to calculate the dementia-related index using brain images and patient information. Model 3 and Model 4 are pre-trained models using brain images and patient information.

Model 3 is a model in which additional patient information (age, gender, and education level) is applied in addition to Model 1. In other words, Model 3 is a model that calculates the dementia-related index using (i) the lateral ventricular and entire extracerebral CSF regions extracted from the brain image and (ii) the patient information (age, gender, and education level). The AUC of Model 3 was 0.829.

Model 4 is a model in which additional patient information (age, gender, and education level) and clinical information (A POE e4) are applied in addition to Model 1. In other words, Model 4 is a model that calculates the dementia-related index using (i) the lateral ventricular and entire extracerebral CSF regions extracted from the brain image, (ii) the patient information (age, gender, and education level), and (iii) the patient clinical information (A POE e4). APOE e4 refers to the genotype information of a dementia-related gene. The AUC of Model 4 was 0.883.

Both Model 3 and Model 4 outperformed Model 1. Therefore, both Model 3 and Model 4 are found to be sufficiently meaningful in diagnosing or predicting dementia.

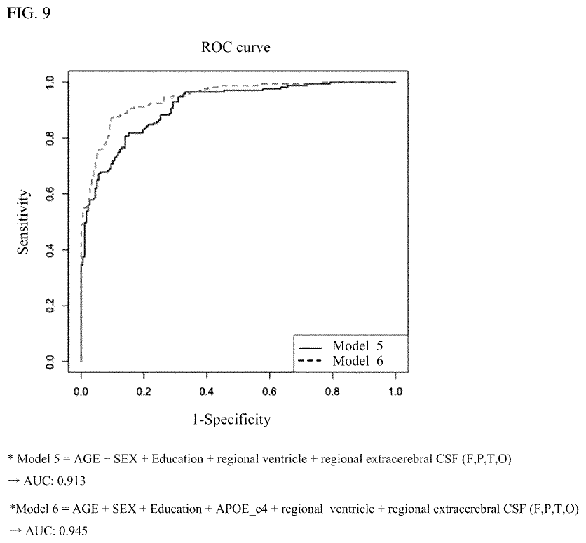

FIG. 9 shows performance evaluation results of classifiers in which patient information is applied to the lateral ventricular and extracerebral CSF subregions. FIG. 9 shows the performance of two models (Model 5 and Model 6). Model 5 and Model 6 are models to calculate the dementia-related index using brain images and patient information. Model 5 and Model 6 are pre-trained models using brain images and patient information.

Model 5 is a model that calculates the dementia-related index using (i) the ventricular subregions and the extracerebral CSF subregions (F, P, T, and O) extracted from the brain image and (ii) the patient information (age, gender, and education level). The AUC of Model 5 was 0.889.

Model 6 is a model that calculates the dementia-related index using (i) the ventricular subregions and the extracerebral CSF subregions (F, P, T, and O) extracted from the brain image, (ii) the patient information (age, gender, and education level), and (iii) the patient clinical information (APOE e4). The AUC of Model 6 was 0.932.

Both Model 5 and Model 6 outperformed Model 2. Therefore, both Model 5 and Model 6 are found to be sufficiently meaningful in diagnosing or predicting dementia.

To sum up, it is found that (i) the entire extracerebral CSF region, (ii) all the subregions of the extracerebral CSF region, (iii) some regions of the subregions of the extracerebral CSF region, (iv) the lateral ventricles+ the entire extracerebral CSF region, (v) the lateral ventricles+ all the subregions of the extracerebral CSF region, (vi) the lateral ventricles+ some regions of the subregions of the extracerebral CSF region, and (vii) the ventricular subregions+at least some regions of the subregions of the extracerebral CSF region are all meaningful as the ROIs associated with the dementia-related index. In the meantime, the classifiers experimentally using the subregions of the extracerebral CSF region as the ROIs slightly outperformed the classifiers using the entire extracerebral CSF region. In addition, the classifiers using only brain MRI also showed sufficiently significant performance. Furthermore, when applying additional information, such as patient information, in addition to brain MRI images, the performance of the classifiers was confirmed to be improved.

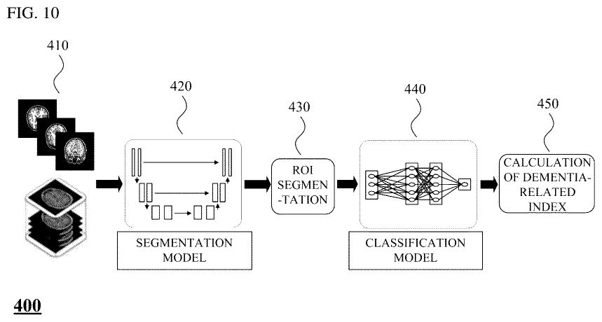

FIG. 10 illustrates an example of a process 400 in which a dementia-related index is calculated using a learning model. FIG. 10 shows a case where the learning model is used in both the process of extracting an ROI from a brain MRI image and the process of predicting a dementia-related index on the basis of the ROI.

The analysis apparatus receives a brain image (M RI image) of a subject (410).

The analysis apparatus may input the received brain image into a pre-trained segmentation model (420). The segmentation model may be implemented as models of various types or structures. For example, the segmentation model may be a model based on U-net. The segmentation model may segment various ROIs depending on the types or learning processes thereof. The segmentation model may segment the ROI from 3D brain images. Alternatively, the segmentation model may segment the ROI from individual 2D slices. As described above, some regions of various regions may be used as the ROI. The segmentation model may be provided in advance for each specific ROI. For example, the segmentation model may be diverse as follows: (i) a model that segments the entire extracerebral CSF region, (ii) a model that segments at least some of the extracerebral CSF subregions, (iii) a model that segments the entire extracerebral CSF region+ the lateral ventricular region, (iv) a model that segments at least some of the extracerebral CSF subregions+ the lateral ventricular region, and the like. The segmentation model may be constructed in advance as a model that segments any one of the various ROIs depending on the learning data and learning processes.

The analysis apparatus may obtain the results (ROI identification) output by the segmentation model (430).

The analysis apparatus inputs the ROI into a pre-trained classification model (440). The classification model is a model that calculates the dementia-related index by inputting images or images and patient information.

The classification model is a machine learning model. Therefore, the classification model may be any one of various types of models. For example, the classification model may be a model implemented using any one of the following methods: decision trees, random forests (RFs), K-nearest neighbors (KNN), Naive Bayes, support vector machines (SV M s), artificial neural networks (ANNs), regression models, and the like. There are also various types of ANN models. For example, the classification model may be a convolutional neural network (CNN)-based model.

The analysis apparatus may obtain the dementia-related index output by the classification model (450). The dementia-related index may be information such as the volume of the ROI, the degree of brain atrophy, or the degree of dementia progression.

The classification model may calculate the dementia-related index using only the ROI. In addition, the classification model may calculate the dementia-related index by inputting the ROI and patient information (age, gender, education level, and the like) into the classification model. Furthermore, the classification model may also calculate the dementia-related index by inputting the ROI, patient information (age, gender, education level, and the like), and clinical information (A POE e4 and the like) into the classification model.

In the meantime, unlike FIG. 10, the learning model may be used in any one of the processes of extracting the ROI or predicting the dementia-related index. In other words, (i) the analysis apparatus may extract the ROI using the segmentation model and estimate the degree of brain atrophy by calculating the volume of the extracted ROI. Calculating the volume and determining the degree of brain atrophy are as described in FIG. 3 or FIG. 4. Alternatively, (ii) the analysis apparatus may extract the ROI using a mask and calculate the dementia-related index by inputting the extracted ROI into the classification model in FIG. 10. The process of extracting the ROI using the mask is as described in FIG. 3 or FIG. 4.

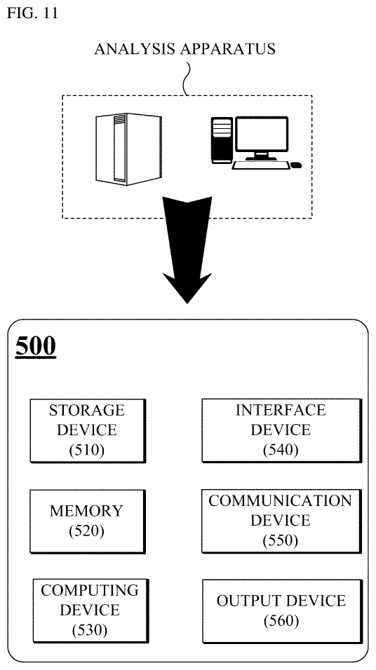

FIG. 11 illustrates an example of an analysis apparatus 500 configured to calculate a dementia-related index. The analysis apparatus 500 corresponds to the analysis apparatus (130 and 140 in FIG. 1) described above. The analysis apparatus 500 may be physically implemented in various forms. For example, the analysis apparatus 500 may have forms of a computer device, such as a PC, a network server, a chipset dedicated to data processing, and the like.

The analysis apparatus 500 may include a storage device 510, a memory 520, a computing device 530, an interface device 540, a communication device 550, and an output device 560.

The storage device 510 may store a brain image (M RI image), which is produced in medical imaging equipment, of a subject.

The storage device 510 may store patient information and clinical information about the subject. The patient information and the clinical information are as described above.

The storage device 510 may store a code or program that calculates the dementia-related index from the brain image.

The storage device 510 may store a mask (or masks) extracted from the brain image.

The storage device 510 may store a segmentation model for extracting an ROI from the brain image. The segmentation model is a pre-trained model.

The storage device 510 may store a classification model that calculates the dementia-related index by receiving the ROI. The classification model is a pre-trained model.

The storage device 510 may store the dementia-related index of the subject.

The memory 520 may store data, information, and the like produced during a process in which the analysis apparatus 500 calculates the dementia-related index from the brain image.

The interface device 540 is a device configured to receive certain commands and data from outside. The interface device 540 may receive the brain image of the subject from a physically connected input device or an external storage device. The interface device 540 may receive patient information and/or clinical information of the subject from a physically connected input device or an external storage device. The interface device 540 may also deliver the dementia-related index calculated on the basis of the brain image to an external object.

The communication device 550 refers to a configuration that receives and transmits certain information via a wired or wireless network. The communication device 550 may receive the brain image of the subject from an external object. The communication device 550 may receive patient information and/or clinical information of the subject from an external object. The communication device 550 may also transmit the dementia-related index calculated on the basis of the brain image to an external object such as a user terminal.

The interface device 540 and the communication device 550 are configurations that exchange certain data from a user or other physical objects and thus may also be comprehensively referred to as input/output devices. In terms of information or data input functions, the interface device 540 and the communication device 550 may also be referred to as input devices.

The output device 560 is a device configured to output certain information. The output device 560 may output an interface required during a data processing process, the brain image, the ROI segmented from the brain image, the dementia-related index calculated on the basis of the ROI, and the like.

The computing device 530 may consistently preprocess the brain image of the subject. The data preprocessing process is as described in FIG. 3. The computing device 530 may produce the mask (or masks) for segmenting the entire brain region and the ROI through the data preprocessing process.

The computing device 530 may segment the entire brain region from the brain image using the mask for the entire brain region. Furthermore, the computing device 530 may segment a target ROI from the entire brain region using the mask for a specific ROI. As described above, the ROI may be diverse. For example, the ROI may be any one of the entire extracerebral CSF region, the extracerebral CSF subregions, the entire extracerebral CSF region+ the lateral ventricular region, and the extracerebral CSF subregions+ the lateral ventricular region.

The computing device 530 may segment the ROI from the received brain image using the segmentation model.

The computing device 530 may calculate the volume or area of the segmented ROI. The computing device 530 may calculate the volume or area of the ROI using any one of the commonly used programs for calculating the volume of the brain region. Furthermore, the computing device 530 may normalize the initially calculated volume of the ROI on the basis of the ICV.

The computing device 530 may estimate the degree of brain atrophy on the basis of the volume or area of the final ROI. Alternatively, the computing device 530 may estimate the degree of dementia of the subject on the basis of the volume or area of the final ROI. Alternatively, the computing device 530 may estimate the degree of dementia of the subject on the basis of the degree of brain atrophy of the subject.

The computing device 530 may calculate the dementia-related index by inputting the ROI into the classification model constructed in advance. In addition, the computing device 530 may calculate the dementia-related index by inputting the ROI and patient information (age, gender, education level, and the like) into the classification model. Furthermore, the computing device 530 may calculate the dementia-related index by inputting the ROI, patient information (age, gender, education level, and the like), and clinical information (A POE e4 and the like) into the classification model.

The computing device 530 may be a device, such as a processor, an application processor (AP), or a program-embedded chip, that processes data and processes certain operations.

In addition, a method of processing the brain image described above, calculating the dementia-related index, or predicting dementia may be implemented using a program (or application) incorporating an executable algorithm that can be run on a computer. This program may be stored in a transitory or non-transitory computer-readable medium and provided.

The non-transitory computer-readable medium refers to a medium that semi-permanently stores data and is readable by a device, other than a medium, such as a register, cache, or memory, that stores data for a short period of time. Specifically, various applications or programs described above may be stored in non-transitory computer-readable media such as a compact disc (CD), a digital versatile disc (DVD), a hard disk, a Blu-ray disc, a Universal Serial Bus (USB), a memory card, a read-only memory (ROM), a programmable ROM (PROM), erasable PROM (EPROM), electrically EPROM (EEPROM), or a flash memory and provided.

The transitory computer-readable medium refers to various types of random-access memory (RAM) such as static RAM (SRAM), dynamic RAM (DRAM), synchronous DRAM (SDRAM), double data rate SDRAM (DDR SDRAM), enhanced SDRAM (ESDRAM), Synclink DRAM (SLDRAM), and Direct Rambus RAM (DRRAM). While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of calculating a dementia-related index based on a volume of an extracerebral cerebrospinal fluid region, the method comprising:
receiving, by an analysis apparatus, a brain image of a subject;
identifying, by the analysis apparatus, a region of interest from the brain image;
calculating, by the analysis apparatus, a volume or area of the region of interest; and
calculating, by the analysis apparatus, a dementia-related index of the subject on the basis of the volume or area,
wherein the region of interest comprises an extracerebral cerebrospinal fluid region.

2. The method of claim 1, wherein the dementia-related index comprises at least one of degree of brain atrophy, presence or absence of dementia, and degree of dementia progression.

3. The method of claim 1, wherein the analysis apparatus identifies the region of interest using a mask produced by preprocessing the brain image.

4. The method of claim 1, wherein the region of interest further comprises a ventricular region, and
the ventricular region comprises lateral ventricles.

5. The method of claim 1, wherein the region of interest is a subregion comprised in the extracerebral cerebrospinal fluid region, and
the subregion comprises at least one of a frontal region, a temporal region, a parietal region, and an occipital region.

6. A method of calculating a dementia-related index based on a volume of an extracerebral cerebrospinal fluid region, the method comprising:
receiving, by an analysis apparatus, a brain image of a subject;
identifying, by the analysis apparatus, a region of interest from the brain image; and
inputting, by the analysis apparatus, the region of interest into a pre-trained learning model to calculate a dementia-related index of the subject,
wherein the region of interest comprises an extracerebral cerebrospinal fluid region.

7. The method of claim 6, wherein the analysis apparatus identifies the region of interest by inputting the brain image into a pre-trained segmentation model.

8. The method of claim 6, wherein the region of interest comprises the entire extracerebral cerebrospinal fluid region and a lateral ventricular region.

9. The method of claim 6, wherein the region of interest is a subregion comprised in the extracerebral cerebrospinal fluid region, and
the subregion comprises at least one of a frontal region, a temporal region, a parietal region, and an occipital region.

10. The method of claim 9, wherein the region of interest further comprises a lateral ventricular region.

11. The method of claim 9, wherein the analysis apparatus further inputs additional information of the subject to the learning model to calculate the dementia-related index, and
the additional information comprises at least one of age, gender, education level, and A POE e4 genotype of the subject.

12. An analysis apparatus configured to calculate a dementia-related index, the analysis apparatus comprising:
an input device configured to receive a brain image of a subject; and
a computing device configured to identify a region of interest from the brain image and calculate a dementia-related index on the basis of the region of interest,
wherein the region of interest comprises an extracerebral cerebrospinal fluid region.

13. The analysis apparatus of claim 12, wherein the computing device identifies the region of interest using a mask produced by preprocessing the brain image.

14. The analysis apparatus of claim 12, wherein the computing device identifies the region of interest by inputting the brain image into a pre-trained segmentation model.

15. The analysis apparatus of claim 12, wherein the computing device calculates the dementia-related index on the basis of a volume or area of the region of interest.

16. The analysis apparatus of claim 12, wherein the computing device calculates the dementia-related index by inputting the region of interest into a pre-trained learning model.

17. The analysis apparatus of claim 12, wherein the region of interest comprises the entire extracerebral cerebrospinal fluid region and a lateral ventricular region.

18. The analysis apparatus of claim 12, wherein the region of interest is a subregion comprised in the extracerebral cerebrospinal fluid region, and
the subregion comprises at least one of a frontal region, a temporal region, a parietal region, and an occipital region.

19. The analysis apparatus of claim 12, wherein the region of interest is a subregion comprised in the extracerebral cerebrospinal fluid region, and
the subregion comprises a lateral ventricular region and at least one of a frontal region, a temporal region, a parietal region, and an occipital region.
